(19) United States
(12) Patent Application Publication
(10) Pub. No.: US 2025/0235398 A1
(43) Pub. Date: Jul. 24, 2025

(54) HIGH DOSAGE INJECTABLE BEVACIZUMAB FORMULATIONS AND METHODS

(71) Applicant: Serán BioScience, LLC, Bend, OR (US)

(72) Inventors: Dan Smithey, Bend, OR (US); Erica Schlesinger, Sisters, OR (US); Mark Kastantin, Bend, OR (US); Cindy Chung, Bend, OR (US); Mary Collins, Bend, OR (US)

(21) Appl. No.: 19/035,749

(22) Filed: Jan. 23, 2025

Related U.S. Application Data
(60) Provisional application No. 63/624,724, filed on Jan. 24, 2024.

(51) Int. Cl. A61K 9/00; A61K 9/10; A61K 9/16; A61K 47/14; C07K 16/22

(52) U.S. Cl. CPC A61K 9/0019; A61K 9/10; A61K 9/1623; A61K 9/1641; A61K 9/1652; A61K 9/1682; A61K 47/14; C07K 16/22

(57) ABSTRACT

Systems and methods are provided for a dried formulation of bevacizumab. The dried formulation includes bevacizumab, a thermal stabilizer, and buffer salts. The bevacizumab is included in the dried formulation in a range of from 50 wt. % to 90 wt. %.

### CROSS-REFERENCE TO RELATED APPLICATIONS

The present application claims priority to U.S. Provisional Application No. 63/624,724 entitled “HIGH DOSAGE INJECTABLE BEVACIZUMAB FORMULATIONS AND METHODS”, filed Jan. 24, 2024. The entire contents of the above identified application(s) is hereby incorporated by reference for all purposes.

### FIELD

The present description relates generally to high dosage injectable formulations of bevacizumab and methods thereof.

### BACKGROUND/SUMMARY

It is not uncommon for some pharmaceutical formulations including certain types of active pharmaceutical ingredients (APIs), such as proteins, to demand delivery by injection. For such pharmaceutical formulations, administration by subcutaneous or intramuscular injection may be preferred over intravenous (IV) administration. Subcutaneous or intramuscular injections may be performed at home, in some examples by using an auto-injector. At home administration improves patient experience and compliance and may also reduce health care costs. A formulation of an active pharmaceutical ingredient, such as a protein, delivered by subcutaneous or intramuscular injection often demands either a high concentration of API and/or a high injected volume to achieve target doses. Additionally or alternatively, an ocular injection may similarly demand the high concentration of API. One approach to increasing a dose concentration in an injectable pharmaceutical formulation is to prepare a high concentration solution. However, high concentration solutions may be limited to concentrations between 100-200 mg/mL before being limited by an increased viscosity and decreased stability. As an alternative, API may be provided as a suspension of microparticles including the API in a vehicle that does not solubilize the microparticles. Such an approach may increase an achievable concentration, but may still be limited by injectability of the resulting suspension due to a large dispensing force created by the high apparent suspension viscosity.

Bevacizumab is conventionally administered by IV. Formulations of solutions for IV administration of bevacizumab conventionally include 25 mg/mL bevacizumab in a phosphate buffered solution along with a stabilizer (trehalose) and a surfactant (polysorbate 20). The formulation is further diluted into 0.9% saline solution before administration. The 25 mg/mL stock solution is stable for up to 2 years at −20° C. and for 45 days at 2-8° C. Conventionally, a dosage of bevacizumab is 5 mg/kg to 15 mg/kg administered by IV infusion every 2-3 weeks. If the conventional IV solution at 25 mg/mL was administered by injection, a volume of 14-42 mL would be needed for each dose. Such volumes are above a conventional threshold for injection volume (e.g., <3 mL). Advanced medical devices and/or delivery technologies which increase expense and complexity for the patient may be demanded to administer such high volumes. A stable formulation which includes bevacizumab at a demanded dosage in a 2 mL volume administered by conventional injection or an auto-injector may increase patient comfort and decrease the overall cost of receiving the medication. Additionally, a dried formulation of bevacizumab may be used to prepare a suspension and/or solution for IV administration or for subcutaneous injections in a hospital setting. The dried bevacizumab may offer advantages over a conventional 25 mg/mL solution in terms of temperature stability, and ease of transportation and storage.

In one example, the issues described above may be at least partially addressed by a dried formulation of bevacizumab, comprising bevacizumab, a thermal stabilizer, and buffer salts, wherein the bevacizumab is included in the dried formulation in a range of from 50 wt. % to 90 wt. %. The dried formulation may be included in a suspension with a non-aqueous carrier at a concentration of between 40 wt. % and 60 wt. %. The suspension of the dried formulation may be injectable through a 27 gauge ½ inch long needle and a 1 mL syringe body at a dispense rate greater than or equal to 0.125 mL/s and with a glide force of less than 100N. In this way, a demanded dosage of bevacizumab may be delivered in volume between 1 mL and 3 mL via an autoinjector. The dried formulation and the suspension may be stable at room temperature and stability may be equivalent to, if not greater than a corresponding 25 mg/mL solution of bevacizumab.

It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

FIG. 1 shows a graph of glide force for administering a suspension as function of tapped density of bevacizumab particles included in the suspension.

FIG. 2A shows a graph comparing glide force for administering a suspension of a dried formulation of bevacizumab at different concentrations.

FIG. 2B shows a graph comparing glide force for administering a suspension of a dried formulation of bevacizumab in propylene glycol dicaprylate/dicaprate at different concentrations.

FIG. 3 shows a graph of % high molecular weight (HMW) species as a function of hold time for a spray solution of bevacizumab.

FIG. 4 shows a graph comparing % HMW species of a spray solution and of a wet spray dried product obtained from the spray solution.

FIG. 5 shows a graph of % HMW species as a function of hold time of the spray dried product in a collection vessel.

FIG. 6 shows a graph comparing % aggregates in a spray dried power and a suspension including the spray dried powder measured over 6 months refrigerated storage.

FIG. 7 shows a flowchart of a method for preparing a dried formulation and an injectable formulation.

### DETAILED DESCRIPTION

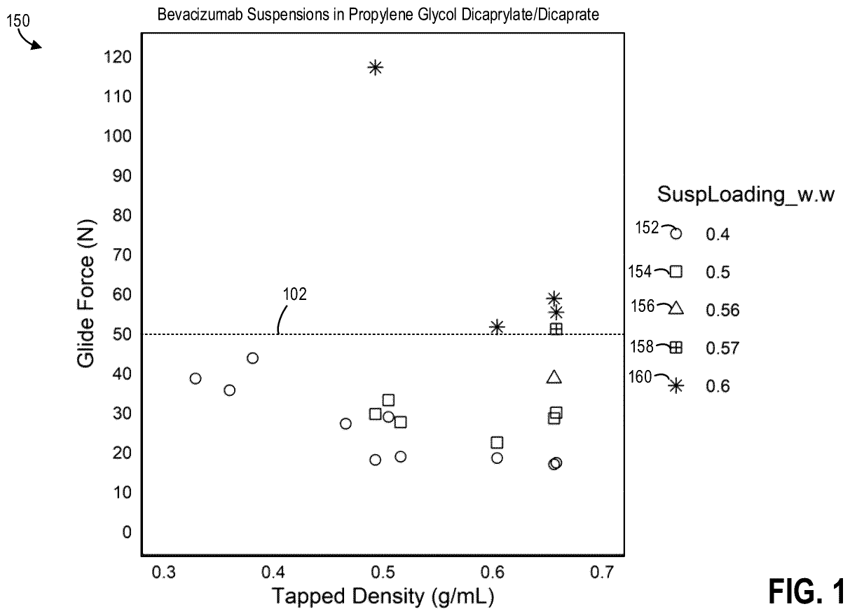

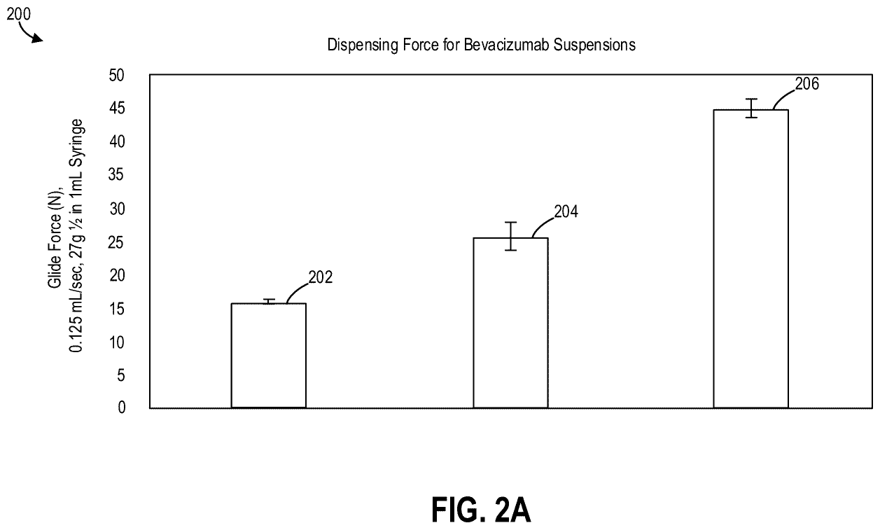

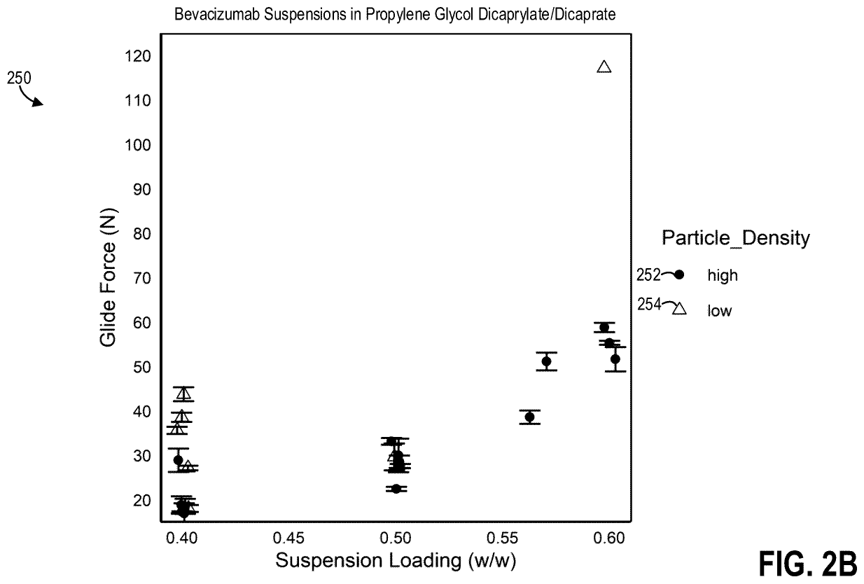

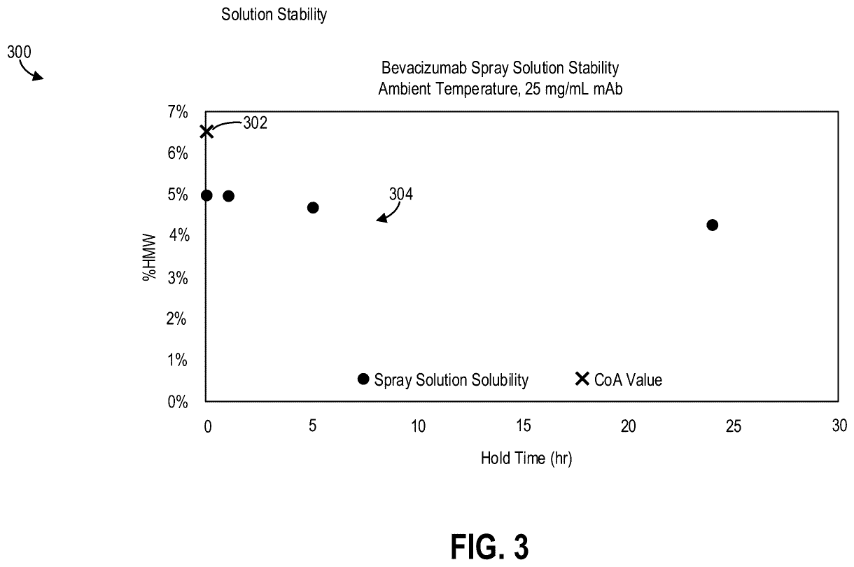

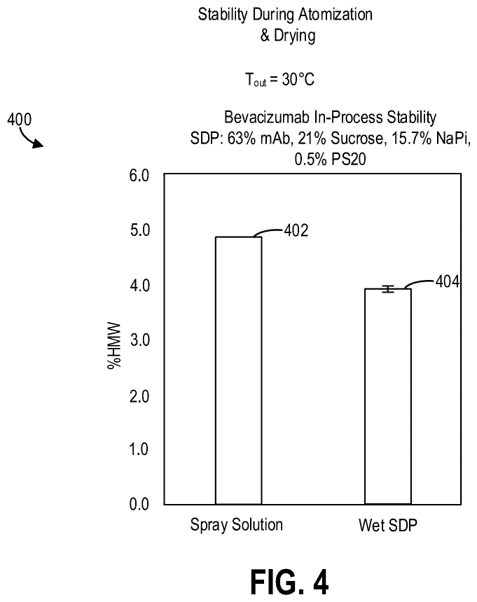

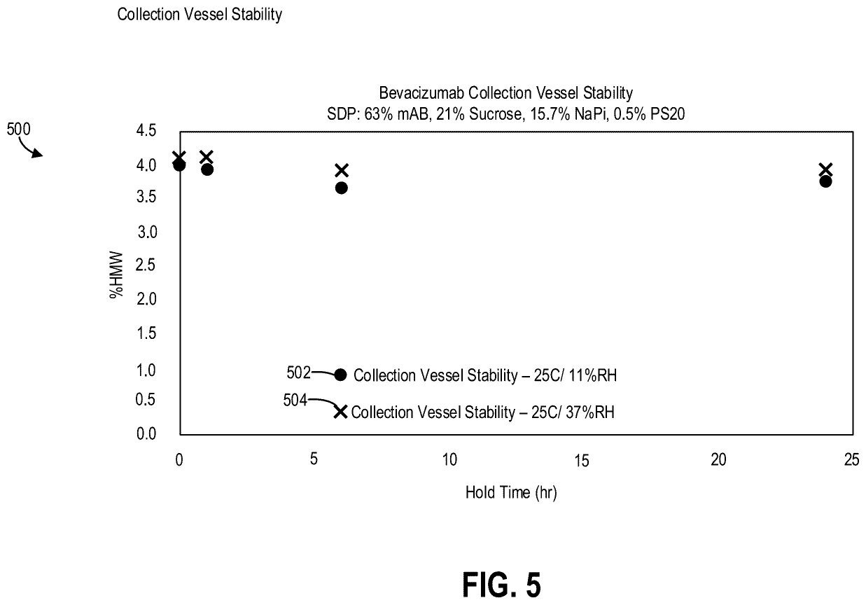

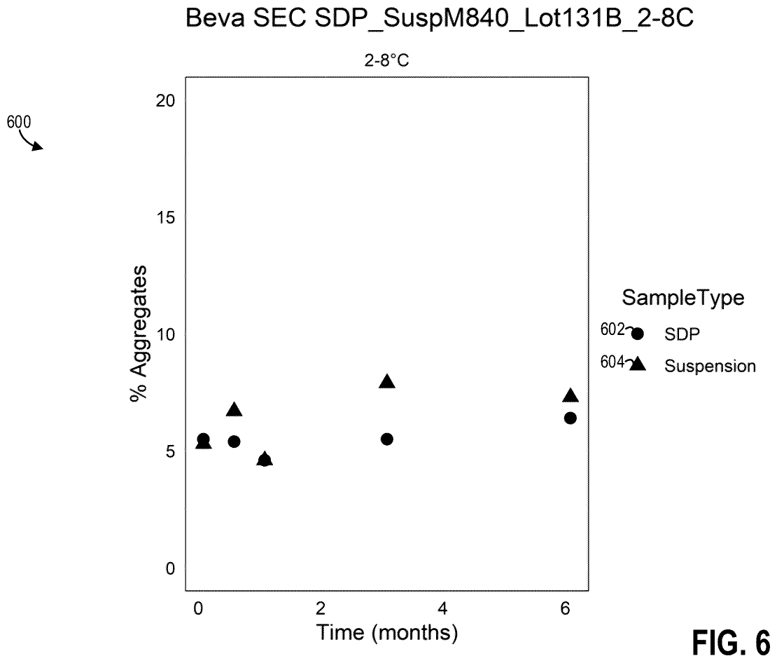

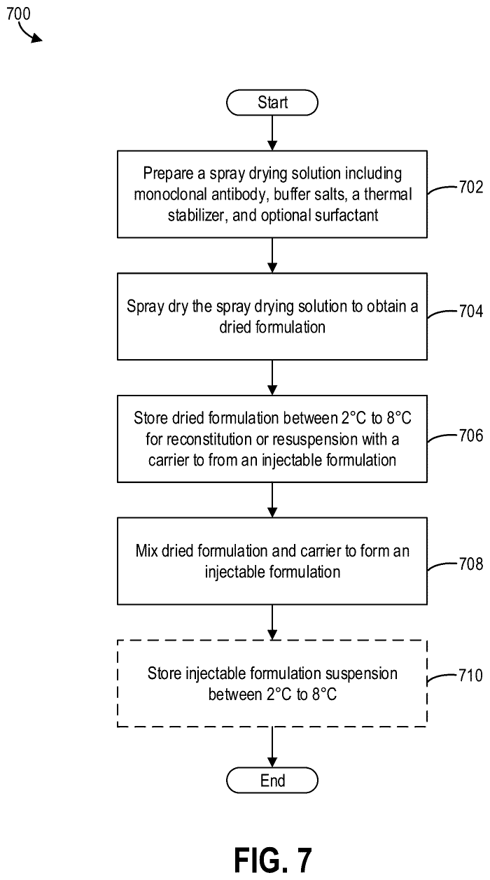

The following description relates to systems and methods for a dried formulation of bevacizumab and an injectable formulation formed from the dried bevacizumab. The injectable formulation may be a suspension in non-aqueous carrier or a solution in aqueous carrier for injection and methods thereof. The following description is relevant to monoclonal antibodies broadly that may target a vascular endothelial growth factor A gene in humans (e.g., anti-VEGF-A antibodies). For example, the monoclonal antibody may comprise complementary determining regions (CDRs) capable of binding human VEGF. Further, the heavy chain CDRs may include the amino acid sequences: GYTFTNYGMN, WINTYTGEPTYAADFKR, and YPHYYGSSHWYFD; and the light chain CDRs may include the amino acid sequences: SASQDISNYLN, FTSSLHS, and QQYSTVPWT. Additionally, the monoclonal antibody may have a weight in a range of 140 kDa to 160 kDa. The suspension may include the monoclonal antibody at a concentration above a threshold concentration for administration of a conventional dose of the monoclonal antibody by a single injection via a conventional syringe or auto-injector. The suspension may include dried particles, the dried particles (e.g., dried formulation) including the monoclonal antibody in addition to other excipients. The dried particles may be above a threshold tapped density, the threshold tapped density be correlated with a resulting glide force demanded to inject the suspension as shown in the graph of FIG. 1. The glide force demanded to inject the suspension may depend on a weight % of the particles in the suspension, a tapped density of the dried particles, as well as an identity of the non-aqueous carrier in which the particles are suspended as shown in FIGS. 2A and 2B. The glide force may further depend on dispensing rate and syringe geometry. When including a monoclonal antibody in a formulation, stability of the monoclonal antibody at each step of the manufacture is of concern. FIG. 3 shows a graph showing stability of a solution used for spray drying the monoclonal antibody. FIG. 4 shows the stability of the monoclonal antibody during spray drying. The graph of FIG. 5 shows short term (e.g., 24 hours) stability of the spray dried product while graph of FIG. 6 compares long term (e.g., up to 6 months) stability of the spray dried product and of the suspension including the spray dried product. A flowchart of a method for preparing the dried formulation, injectable formation, and administering the injectable formulation is shown in FIG. 7.

Table 1 below compares a non-limiting example injectable formulation including a suspension of dried bevacizumab particles in a non-aqueous carrier with a conventional bevacizumab formulation for infusion. In further examples, the injectable formulation may be a solution prepared by reconstituting the dried bevacizumab particles. The solution may be prepared according to the type of injection. For example, the solution may be prepared to be an IV infusion, a subcutaneous injection, an intramuscular injection, or an ocular injection.

TABLE 1
Comparing properties of bevacizumab suspension and solution

| | | Bevacizumab Solution |
|---|---|---|
| | Bevacizumab Suspension | (Prior Art) |
| Delivery | Autoinjector/Subcutaneous | IV infusion |
| Dose Range | 500 mg-1500 mg | 5 mg/kg-15 mg/kg |
| | (assuming ~70 kg avg. | |
| | human) | |
| Dosing Regimen | Weekly or biweekly | Every 2-3 weeks |
| Concentration | 400 mg/mL | 25 mg/mL |
| Delivered Volume | 0.625 mL-1.88 mL | N/A |
| | (weekly) | |
| | 1.25 mL-3.75 mL | |
| | (biweekly) | |
| Particle Size Range | 1-50 μm | N/A |
| Injection Time | 1 mL/10 s-1 mL/8 s | N/A |
| Max Dispense Force | <50N, <100N | N/A |
| Stability | Min: 2-8° C. for up | −20° C. for 2 years |
| | to 2 years | 2-8° C. for 45 days |
| | | up to 5 freeze/thaw |
| | | cycles |

In addition to being deliverable by autoinjector, the bevacizumab suspension may also have increased stability over the solution. Concentrations in a range of 125 mg/mL-600 mg/mL may be selected based on a demanded dosage to be delivered in a volume range of 1 mL-4 mL. In some examples, the volume range may be from 1 mL to 3 mL. In some examples, an injection volume may be less than 3 mL or less than 1 mL. In further examples, an injection volume may be in a range of 0.5 mL to 3 mL. In some examples, the concentration of bevacizumab in suspension may be in a range of 100 mg/mL to 600 mg/mL. In a non-limiting example, a lower concentration of bevacizumab may be demanded and the concentration in the suspension may be in a range of 100 mg/mL to 200 mg/mL. In further examples, the bevacizumab in suspension may be in a range of 125 mg/mL to 600 mg/mL. In one example, the concentration of bevacizumab in suspension may be 400 mg/mL. A concentration range may be limited by a demand that the suspension has a viscosity which results in a max dispense force of <50N or <100N when injecting the suspension of the demanded concentration of particles through a conventional (e.g., 27 gauge, ½ inch long) needle from a 1 to 3 mL syringe at a rate of 1 mL/8 s or at a rate of 1 mL/10 s. A syringe plunger rod may be the same for syringe barrels from 1 to 3 mL. In one example, a max dispense force may be less than 20N. In one example, the rate may be 1 mL/8 s.

The viscosity of a bevacizumab suspension may depend on both concentration of the suspended particles and the physical properties of the suspended particles. The particles including bevacizumab may be formed by a drying process. In some examples the drying process may be precipitation or lyophilization. In alternate examples, the drying process may be spray drying. Spray drying has an advantage of being a continuous process which is well adapted to large scale production of dried powders. The spray drying process has been demonstrated to be suitable for manufacture of dried bevacizumab formulations without impact to product quality. In one example, the drying process may be performed at a temperature below an atmospheric pressure boiling point of the spray drying solution. A low temperature spray drying process may be desired due to a thermal instability of the bevacizumab. Additionally, or alternatively, the drying process may be a vacuum spray drying process.

FIG. 1 shows a graph 150 of glide force of a suspension including dried particles comprised of bevacizumab suspended at 40 wt. % in a propylene glycol dicaprylate/dicaprate carrier (e.g., Miglyol® 840) as a function of tapped density of a collection of the spray dried particles. Glide force is measured by dispensing 1 mL of the suspension through a 27 gauge ½ inch long needle at a rate of 1 mL/8 s. For the experimental measurements shown in FIGS. 1A-2B the syringe used is manufactured by Becton, Dickson, & Company. The barrel is a Neopak™ 1MLL27G1/2-5BSTW and the plunger rod is a Hypak™ PR1MLL.

A line 102 corresponds to an example of a threshold glide force of 50N for administrating an injection. In alternate examples, the threshold glide force may be 100N. In some examples, a minimum tapped density of dried powders of bevacizumab may be selected. For example, a threshold tapped density of a dried formulation of bevacizumab may be 0.3 g/mL. In some examples, the threshold tapped density may be 0.4 g/mL. In further examples, the threshold tapped density may be 0.5 g/mL or 0.6 g/mL. In some examples, the threshold tapped density may increase as a demanded % weight of the dried formulation in the suspension increases, as described further below.

A first set of open circle data points 152 corresponds to suspensions loaded at 40% by weight dried particles. A second set of open square data points 154 corresponds to suspensions loaded at 50% by weight dried particles. A third set of open triangle data points 156 corresponds to suspensions loaded at 56% by weight dried particles. A fourth set of crossed square data points 158 corresponds to suspensions loaded at 57% by weight dried particles. A fifth set of star data points 160 corresponds to suspensions loaded at 60% by weight dried particles.

As shown in FIG. 1, suspensions prepared at 40% and 50% spray dried particles by weight result in suspensions which are injectable at glide forces at or below 50N even at low tapped densities, such as at 0.3 g/mL. As loading of the suspension is increased above 50% by weight a higher tapped density may be demanded to maintain a glide force at or below 50N. For example, for suspensions prepared at 60% by weight dried particles (star data points 160), at a tapped density of 0.5 g/mL the glide force is more than double the 50N threshold, but the glide force is lowered to nearly the 50N threshold for tapped densities increased to 0.65 g/mL. Further, the glide force may be decreased below 50N by lowering solids loading from 60% by weight to 56% by weight (open triangle data points 156). In this way, increasing tapped density of the dried powder enable loading of suspensions above 50% by weight.

Dried particles (e.g., a dried formulation) may include bevacizumab in addition to other excipients. The additional excipients may include a thermal stabilizer, buffer salt, and a surfactant, and may be selected to increase the stability of the active ingredient (e.g., bevacizumab) both during the drying process and during storage, or to achieve specific design features of the dried particle. Formulations may also be comprised of an amino acid. Examples of formulations are shown in table 2 below. Formulation numbers are given to aid in further discussion. Additionally, ranges of concentrations and non-limiting examples of components of the dried formulation are given in table 3.

TABLE 2
Relative composition by weight of components
of a particle, Bev. = bevacizumab

| | Rel. | | | Rel. | Rel. |
|---|---|---|---|---|---|
| | protein by | Rel. thermal | | surfactant by | buffer salt |
| | weight | stabilizer by weight | | weight | by weight |
| 1 | 62.85 (IgG) | 20.94 | (sucrose) | 0.50 | 15.70 |
| 2 | 62.85 (Bev.) | 20.94 | (trehalose) | 0.50 | 15.71 |
| 3 | 73.05 (Bev.) | 20.94 | (proline) | 0.50 | 15.71 |
| 4 | 73.05 (Bev.) | 8.12 | (sucrose) | 0.58 | 18.25 |
| 5 | 73.05 (Bev.) | 8.12 | (trehalose) | 0.58 | 18.25 |
| 6 | 73.05 (Bev.) | 8.12 | (proline) | 0.58 | 18.25 |

TABLE 3
Examples of formulation components and compositions

| Component | | Ranges |
|---|---|---|
| Type | Non-limiting Examples | (w/w solid) |
| Active | Bevacizumab or equivalent | 50%-90% |
| Thermal | Sucrose, Trehalose, Proline, HPBCD, | 5%-50% |
| Stabilizer(s) | cyclodextrins, dextrins, dextrans. | |
| Buffer | Sodium Phosphate, Potassium Phosphate, | 1%-20% |
| | Phosphoric Acid, Sodium Citrate, Citric | |
| | Acid, Histidine, Sodium Succinate, | |
| | Succinic Acid. | |
| Surfactant | PS20, PS80, PEG, Poloxamer 188, | 0%-5% |
| | Poloxamer 407 | |

In one example, the non-volatile components of a solution used to prepare a dried formulation are each included homogenously in the dried particle. For this reason, it is assumed that the ratio by weight of components in the dried formulation corresponds to the ratios in the preparation solution and in the dried particle. A thermal stabilizer may be included in the formulation to stabilize bevacizumab during drying and storage. In some examples, the formulation may include more than one and/or a mixture of different thermal stabilizers. The thermal stabilizers may be configured to protect a protein from thermal degradation. As non-limiting examples, the thermal stabilizer may be one or more of a sugar, amino acid, dextrins, dextrans, and cyclodextrans. In some examples, the thermal stabilizer may be one or more of sucrose, trehalose, proline, and hydroxypropyl-β-cyclodextran (HPBCD). In some examples, the thermal stabilizer may be included in the dried formulation in a range of from 5 wt. % to 50 wt. %. The formulation may also include a surfactant. In some embodiments, the surfactant may be a hydrophilic surfactant approved for an injectable pharmaceutical formulation. In some examples, the surfactant may be one or more of polysorbate 20 (PS20), polysorbate 80 (PS80), or poly(ethyleneglycol)(PEG), poloxamer 188, poloxamer 407 or the like. In some examples, the dried formulation may include the surfactant in a range of from 0 wt. % to 5 wt. %. In some examples, a ratio of monoclonal antibody to surfactant may be 125:1. In some examples, the formulation may include buffer salts. The buffer salts may include one or more of sodium phosphate, potassium phosphate, phosphoric acid, sodium citrate, citric acid, histidine, sodium succinate, succinic acid, or the like. In some examples the buffer salts may be included in range of from 1 wt. % to 20 wt. %. In some examples, a ratio of monoclonal antibody to buffer salts may be 4:1. In some examples, a level of buffer salt may be reduced without altering desired physical/chemical properties of the dried formulation. In one example, a ratio of monoclonal antibody to buffer salts may be >4:1. In some examples, the buffer salts may not be included in the dried formulation but removing or reducing the buffer salts may demand an extra manufacturing step, thereby increasing a cost and manufacturing time of the formulation. In one example, bevacizumab may be included in the dried formulation at greater than or equal to 50% by weight. In some examples, bevacizumab may be included in the dried formulation in greater than or equal to 75% by weight. In alternate examples, bevacizumab may be included in the formulation in a weight range of from 50% to 75% by weight. In further examples, bevacizumab may be included in the formulation in a range of rom 50 wt. % to 90 wt. %.

In some examples, the dried particles of the dried formulations described above may be included in suspension in a non-aqueous carrier. The non-aqueous carrier may be a liquid precedented for injectable pharmaceutical formulations having a viscosity suitable for injectable formulations. In some examples, the non-aqueous carrier may be one or more of ethyl oleate, propylene glycol dicaprylate/dicaprate, caprylic/capric triglyceride, triacetin, benzyl benzoate, or the like. The suspension may include the dried formulation at a wt. % of greater than or equal to 40%. In further examples, the suspension may include the dried formulation at a wt. % in a range of from 40% to 60%. In further examples, the suspension may include the dried formulation at wt. % in a range of from 40% to 70%. In some examples, the suspension may include the dried formulation at a wt. % of greater than or equal to 20%. If some examples, the suspension may include the dried formation at a wt. % in a range of from 20% to 60%. In some examples, the suspension may include the dried formulation at a wt. % in a range of from 20% to 70%.

Turning now to FIG. 2A, it shows a graph 200 of glide force measured for 1 mL of a suspension dispensed through a 27 gauge ½ include long needle at a rate of 1 mL/8 s. Each suspension was prepared using powder corresponding to formulation #2 of table 2. A first column 202 corresponds to glide force of a suspension including 40 wt. % dried particles in propylene glycol dicaprylate/dicaprate. 40 wt. % of dried particles of formulation #2 corresponds to 285 mg bevacizumab/mL. A second column 204 corresponds to glide force of a suspension including 50 wt. % of dried particles of formulation #2 in propylene glycol dicaprylate/dicaprate. 50 wt. % of dried particles of formulation #2 corresponds to a concentration of 372 mg bevacizumab/mL. A third column 206 corresponds to glide force of a suspension of 60 wt. % dried particles of formulation #2 suspended in ethyl oleate. 60 wt. % of dried particles of formulation #2 corresponds to a concentration of 459 mg bevacizumab/mL. As shown in graph 200, glide force increases as wt. % of dried particles included in the suspension increases. Each of the first column 202, second column 204, and third column 206 correspond to a glide force less than 50N. In this way, graph 200 shows that a suspension of dried particles including bevacizumab in a concentration demanded for injection may be injectable.

FIG. 2B shows a graph 250 of glide force as a function of suspension loading as a w/w fraction. Suspensions are prepared and dispensed as described above with respect to FIG. 2A. Graph 250 shows glide force of suspensions of bevacizumab prepared in propylene glycol dicaprylate/dicaprate. Graph 250 further compares the glide force measured for suspensions prepared with particles having a tapped density greater than 0.5 g/mL as shown by closed circle data points 252 and for suspensions prepared with particles having a tapped density less than 0.5 g/mL as shown by open triangle data points 254.

As shown in FIG. 2B, for low suspension loading (e.g., less than or equal to 0.5 w/w), glide force for suspensions including particles having a tapped density greater than 0.5 g/mL is similar to the glide force for suspension including particles having tapped density less than 0.5 g/mL. As suspension load increases, the difference between the two types of suspensions increases. For example, a suspension at a loading of 0.6 w/w with particles having tapped density less than 0.5 g/mL may demand a glide force 120N. Further, about 0.56 w/w to 0.57 w/w may be a suspensions loading threshold for dispensing suspensions at a glide force of less than or equal to 50N for particles with a high tapped density. In some examples, an autoinjector may have a threshold dispensing of above 50N, for example 100N, and suspension loading may be increased and a glide force above 50N may be acceptable.

In addition to injectability of the final suspension, the formulation of bevacizumab may also be stable throughout the production process and in a final product form as a suspension. A production process may include preparation of a solution that is a precursor to the dried formulation. In one example, the solution may be a spray solution adapted to form particles by spray drying. In some examples, the solution may be adapted to spray drying at temperatures below an atmospheric pressure boiling point of the spray solution. In some examples, the solution may be adapted to spray drying at reduced pressures. Once dried, the particles are collected and then suspended in a non-aqueous carrier.

Turning now to FIG. 3, a graph 300 is shown of % high molecular weight (HMW) species as a function of hold time under open conditions when the solution temperature is at equilibrium with the environment. % HMW species may be measured by size exclusion chromatography. An increase in % HMW species may indicate aggregation and therefore decrease in potency of a monoclonal antibody. For this reason, % HMW species may be monitored to determine stability of a composition including monoclonal antibodies. % HMW may also be expressed as % aggregates. In some examples, stability of bevacizumab may also be determined by measuring charged variants via IEX or iCE, measuring visible and sub-visible particulates, and/or by functional assays such as binding assays. A data point 302 at hold time=0 on graph 300 corresponds to a % HMW species for the bevacizumab as supplied by a manufacturer. Data points 304 correspond to a measured % HMW species of a spray solution including bevacizumab at a concentration of 25 mg/mL used to prepare dried particles including bevacizumab held ambient temperature. Data points 304 show that % HMW species of the spray solution does not increase compared to an expected value for bevacizumab as supplied by the manufacturer. Additionally, data points 304 show that the % HMW species of the spray solution do not increase after 24 hours, indicating the spray solution is stable for at least 1 day.

Turning now to FIG. 4, it shows a graph comparing % HMW species of a spray solution before drying and a % HMW species of the wet, spray dried particles obtained from the spray drier collection vessel. The spray dried particles are collected at a spray dryer outlet temperature of 30° C. The spray dried particles include 63% by weight bevacizumab, 21% by weight sucrose, 15.7% by weight sodium phosphate, and 0.5% by weight polysorbate 20. A first column 402 corresponds to the % HMW species of the spray solution and a second column 404 corresponds to a % HMW species of the wet spray dried particles. As shown in graph 400, spray drying of the spray solution does not cause degradation of bevacizumab by aggregation.

During conventional manufacturing, it may not be feasible or at least not convenient to remove spray dried particles immediately after collection in the collection vessel. For example, spray dried particles may remain in a collection vessel until spray drying of an entire batch of spray solution is finished. For this reason, stability of the spray dried particles in a collection vessel is shown in FIG. 5. FIG. 5 shows a graph 500 of % HMW species of spray dried particles held at conditions relevant to the collection vessel as a function of time. The spray dried particles may be the same formulation as the spray dried particles of FIG. 4 and may be held under conditions where the particles are equilibrated to conditions (temperature and moisture) that are comparable to the outlet conditions for relevant spray drying processes. First data points 502 (circles) correspond to spray dried particles held in a collection vessel at 25° C. and 11% relative humidity (RH). Second data points 504 (crosses) correspond to spray dried particles held in a collection vessel at 25° C. and 37% RH. Graph 400 shows that no significant aggregation of bevacizumab is observed while spray dried particles are held in the collection vessel for up to 24 hours, even when humidity is increased to 37%. In this way, ease of manufacturing may be increased by allowing for spray dried particles to be collected and/or held for a least a day before a suspension including the particles is prepared.

In some examples, the suspension may be refrigerated (e.g., 2° C.-° 8 C) for long term (e.g., up to 2 years) storage. Refrigerated storage may be more convenient and cost efficient compared to sub 0° C. storage demanded for the conventional bevacizumab solution. In some examples, a formulation of spray dried particles may be provided to a healthcare professional as a spray dried powder. The spray dried powder may be dissolved in an aqueous solution to provide a solution with a concentration of 25 mg/mL in a healthcare setting and further diluted in saline before administration. By delivering bevacizumab as spray dried particles instead of a 25 mg/mL solution, a refrigerated supply chain may be used in place of the more logistically complicated sub 0° C. supply chain. In some examples, supplying the bevacizumab as spray dried particles may allow a health care professional to prepare a solution at a concentration higher than 25 mg/mL to be administered subcutaneously at an intermediate concentration, between that of an IV administration and that of an autoinjector.

Turning now to FIG. 6 a graph 600 is shown of % aggregates (e.g., % HMW) as a function of storage time under refrigeration conditions for both spray dried particles and a suspension including the spray dried particles. The spray dried particles include bevacizumab and the suspension is prepared using propylene glycol dicaprylate/dicaprate as the non-aqueous vehicle loaded with the spray dried particles at 50% w/w (e.g., 50 wt. %). Refrigeration conditions corresponds to storage at a temperature in a range of from 2° C. to 8° C. Circle data points 602 corresponds to the spray dried particles and triangle data points 604 correspond to the suspension. As shown in FIG. 6, the percent aggregates in both the suspension and spray dried particles remains below 10% for six months.

A dried formulation of a monoclonal antibody is provided. The dried formulation includes a monoclonal antibody, such as bevacizumab, at a concentration which may be therapeutically relevant for preparation of suspensions for subcutaneous or intramuscular injection. The dried formulation may be above a threshold tapped density and may be included in a suspension at up to 60 wt. % without compromising an injectability of the suspension. In some examples, the dried formulation may be prepared by spray drying and bevacizumab may be stable throughout the spray drying process. In this way, a dried formulation that may be included in an injectable suspension is provided.

Further, methods of preparing the dried formulation are provided, including by spray drying. The method may include determining a tapped density of the spray dried powder to predict a glide force demanded for a resulting suspension. Additionally, methods of preparing and administering the dried formulation are provided. The dried formulation may be administered in a plurality of routes, including as a suspension in a non-aqueous carrier and as a solution in an aqueous carrier.

An example of a method 700 for preparation and use of an injectable formulation including a monoclonal antibody, such as bevacizumab is shown in FIG. 7. At 702, method 700 includes preparing a spray drying solution (e.g., spray solution) including monoclonal antibody, buffer salts, and a thermal stabilizer. Preparing the spray drying solution may include mixing the components of the spray drying solution in a liquid phase until homogeneously distributed. In some examples, the components of the spray drying solution may be soluble in the liquid phase and may form a solution when mixed. The monoclonal antibody may be bevacizumab. The buffer salts and thermal stabilizer may be included at concentrations described above with respect to Tables 2 and 3. Further, the spray drying solution may optionally include a surfactant as discussed above with respect to Tables 2 and 3.

At 704, method 700 includes spray drying the spray drying solution to obtain a dried formulation. The dried formulation may include microparticles comprised of the components of the spray drying solution as discussed above. Spray drying may include spray drying at a temperature below the atmospheric pressure boiling point of the liquid phase of the spray drying solution. In some examples, spray drying may include spray drying under vacuum. Spray drying may include adjusting spray drying parameters (e.g., spray drying solution concentrations and Tout) to obtain a dried formulation having a tapped density at or above a threshold tapped density. In some examples, the tapped density may be greater than or equal to 0.4 g/mL. In further examples, the tapped density may be greater than or equal to 0.5 g/mL. In further example, the tapped density may be greater than or equal to 0.6 g/mL.

At 706, method 700 optionally includes storing the dried formulation between 2° C. and 8° C. for reconstitution or resuspension with a carrier to form an injectable formulation. The injectable formulation may be for one or more of intravenous, subcutaneous, intramuscular, and ocular injection. Storing may include storing for less than or equal to six months or less than or equal to two years with less than 10% agglomeration of spray dried formulation.

At 708, method 700 includes mixing the dried formulation and a carrier to form an injectable formulation. Mixing may include stirring or otherwise agitating the dried formulation and carrier until homogenously combined. In some examples, the carrier may be an aqueous carrier and mixing may include reconstituting the dried formulation to form a solution of the dried formulation and be referred to as an injection formulation solution. In alternate examples, the carrier may be non-aqueous and mixing may include resuspending the dried formulation to form a suspension of the dried formulation and may be referred to as an injectable formulation suspension. Relative concentration of the monoclonal antibody (e.g., bevacizumab) in the injectable formulation may vary based on a type of injectable formulation as discussed further below.

For example, an injectable formulation solution may include bevacizumab or other similar monoclonal antibody at a concentration in a range of from <1 mg/mL up to 150 mg/mL. In some examples, the injectable formulation solution is an intravenous injection and the injectable formulation solution may include bevacizumab or other monoclonal antibody at a concentration of >0 mg/mL and ≤150 mg/mL. For example ≥0.1 mg/mL to ≤150 mg/mL or ≥0.25 mg/mL to ≤150 mg/mL. In alternate examples, the injectable formulation solution may for one or more of ocular injection, subcutaneous injection, and intramuscular injection, and the concentration of the bevacizumab or other monoclonal antibody may be in a range of from 100 mg/mL to 150 mg/mL. In examples where mixing forms the injectable formulation suspension, the injectable formulation may be for an intramuscular and/or subcutaneous injection and a concentration of the bevacizumab or other monoclonal antibody may be in a range of from 125 mg/mL to 600 mg/mL. Due to lower volumes of injection for intramuscular and/or subcutaneous injection a demand of concentration of bevacizumab is higher. Reconstituting dried powder as described in method 700 may help overcome long term storage consideration for products that are otherwise stable enough for a same-day or next-day preparation and injection.

At 710, method 700, in examples where the carrier at step 708 is non-aqueous, optionally includes storing the injectable formulation suspension between 2° C. and 8° C. Storing may include storing for less than or equal to 6 months or less than or equal to two years with less than 10% agglomeration of the dried formulation microparticles in the suspension. Method 700 ends.

The technical effect of the methods described herein are to produce a high concentration, stable bevacizumab material. The material may be in the form of dried formulation (e.g., spray dried particles) or a high concentration injectable formulation suspension for subcutaneous and/or intramuscular injection. Spray drying particles to have a high (e.g., greater than 0.4 g/mL) tapped density may result in injectable formulation suspension having a decreased injection glide force. Increasing tapped density of the dried formulation may result in increasing a loading of the dried formulation in the injectable formulation suspension while maintaining an injection glide force below a threshold achievable by an auto-injector. Further the stability of the dried formulation may enable storage and subsequent reconstitution as an injectable formulation solution at the desired time of administration.

The disclosure also provides support for a dried formulation, comprising, bevacizumab, a thermal stabilizer, and buffer salts, wherein the bevacizumab is included in the dried formulation in range of from 50 wt. % to 90 wt. %. In a first example of the system, the dried formulation further comprises a surfactant. In a second example of the system, optionally including the first example, the surfactant is one or more of polysorbate 20, polysorbate 80, poly(ethylene glycol), poloxamer 188, and poloxamer 407. In a third example of the system, optionally including one or both of the first and second examples, the thermal stabilizer is one or more of sucrose, trehalose, proline, cyclodextrin, dextrins, or dextrans. In a fourth example of the system, optionally including one or more or each of the first through third examples, the bevacizumab is included in a range of from 50 wt. % to 75 wt. % In a fifth example of the system, optionally including one or more or each of the first through fourth examples, the thermal stabilizer is included in a range of from 5 wt. % to 50 wt. %. In a sixth example of the system, optionally including one or more or each of the first through fifth examples, an injectable formulation includes the dried formulation and a carrier, wherein a concentration of bevacizumab in the injectable formulation is in a range of from 125 mg/mL to 600 mg/mL.

The disclosure also provides support for a suspension for injection of bevacizumab, comprising: microparticles comprised of bevacizumab in a range of 50 wt. % to 90 wt. %, and a non-aqueous carrier, wherein a concentration of bevacizumab in the suspension is in a range of 125 mg/ml to 600 mg/ml. In a first example of the system, a percent aggregates of the suspension is less than 10% for after storage in a temperature range of from 2° C. to 8° C. for six months. In a second example of the system, optionally including the first example, the non-aqueous carrier is one or more of ethyl oleate, triacetin, benzyl benzoate, propylene glycol dicaprylate/dicaprate, or caprylic/capric triglyceride. In a third example of the system, optionally including one or both of the first and second examples, a tapped density of the microparticles is greater than or equal to 0.4 g/mL. In a fourth example of the system, optionally including one or more or each of the first through third examples, the suspension is injectable through a 27 gauge ½ inch long needle at a rate of 1 mL/8 s using a force of less than 100N. In a fifth example of the system, optionally including one or more or each of the first through fourth examples, the suspension is configured for one or more of a subcutaneous and intramuscular injection.

The disclosure also provides support for a method of preparing an injectable formulation of bevacizumab, comprising: preparing a spray drying solution including the bevacizumab, buffer salts, and a thermal stabilizer, spray drying the spray drying solution to obtain a dried formulation comprising bevacizumab in a range of from 50 wt. % to 90 wt. %, storing the dried formulation for reconstitution or resuspension with a carrier to form the injectable formulation for one or more of a subcutaneous, intramuscular, ocular, and intravenous injection. In a first example of the method, the carrier is non-aqueous and the injectable formulation is a suspension of the dried formulation and the injectable formulation comprises the dried formulation in a range of from 20% to 60% by weight. In a second example of the method, optionally including the first example, the method further comprises: storing the dried formulation for less than or equal to two years at a temperature in a range of 2° C. to 8° C. In a third example of the method, optionally including one or both of the first and second examples, the carrier is aqueous and the injectable formulation is a solution of the dried formulation. In a fourth example of the method, optionally including one or more or each of the first through third examples, the injectable formulation is for an intravenous injection and a concentration of bevacizumab in the injectable formulation is ≥0.1 mg/mL and ≤150 mg/mL. In a fifth example of the method, optionally including one or more or each of the first through fourth examples, the injectable formulation is for one or more of an ocular injection, subcutaneous injection, and intramuscular injection, and a concentration of bevacizumab in the injectable formulation is in a range of from 100 mg/mL to 150 mg/mL. In a sixth example of the method, optionally including one or more or each of the first through fifth examples, a tapped density of the dried formulation greater than or equal to 0.4 g/mL.

The following claims particularly point out certain combinations and sub-combinations regarded as novel and non-obvious. These claims may refer to “an” element or “a first” element or the equivalent thereof. Such claims should be understood to include incorporation of one or more such elements, neither requiring nor excluding two or more such elements. Other combinations and sub-combinations of the disclosed features, functions, elements, and/or properties may be claimed through amendment of the present claims or through presentation of new claims in this or a related application. Such claims, whether broader, narrower, equal, or different in scope to the original claims, also are regarded as included within the subject matter of the present disclosure.

1. A dried formulation, comprising:
bevacizumab;
a thermal stabilizer; and
buffer salts, wherein the bevacizumab is included in the dried formulation in range of from 50 wt. % to 90 wt. %.

2. The dried formulation of claim 1, wherein the dried formulation further comprises a surfactant.

3. The dried formulation of claim 2, wherein the surfactant is one or more of polysorbate 20, polysorbate 80, poly(ethylene glycol), poloxamer 188, and poloxamer 407.

4. The dried formulation of claim 1, wherein the thermal stabilizer is one or more of sucrose, trehalose, proline, cyclodextrin, dextrins, or dextrans.

5. The dried formulation of claim 1, wherein the bevacizumab is included in a range of from 50 wt. % to 75 wt. %.

6. The dried formulation of claim 1, wherein the thermal stabilizer is included in a range of from 5 wt. % to 50 wt. %.

7. The dried formulation of claim 1, wherein an injectable formulation includes the dried formulation and a carrier, wherein a concentration of bevacizumab in the injectable formulation is in a range of from 125 mg/mL to 600 mg/mL.

8. A suspension for injection of bevacizumab, comprising:
microparticles comprised of bevacizumab in a range of 50 wt. % to 90 wt. %; and
a non-aqueous carrier, wherein a concentration of bevacizumab in the suspension is in a range of 125 mg/ml to 600 mg/ml.

9. The suspension of claim 8, wherein a percent aggregates of the suspension is less than 10% for after storage in a temperature range of from 2° C. to 8° C. for six months.

10. The suspension of claim 8, wherein the non-aqueous carrier is one or more of ethyl oleate, triacetin, benzyl benzoate, propylene glycol dicaprylate/dicaprate, or caprylic/capric triglyceride.

11. The suspension of claim 8, wherein a tapped density of the microparticles is greater than or equal to 0.4 g/mL.

12. The suspension of claim 8, wherein the suspension is injectable through a 27 gauge ½ inch long needle at a rate of 1 mL/8 s using a force of less than 100N.

13. The suspension of claim 8, wherein the suspension is configured for one or more of a subcutaneous and intramuscular injection.

14. A method of preparing an injectable formulation of bevacizumab, comprising:
preparing a spray drying solution including the bevacizumab, buffer salts, and a thermal stabilizer;
spray drying the spray drying solution to obtain a dried formulation comprising bevacizumab in a range of from 50 wt. % to 90 wt. %; and
storing the dried formulation for reconstitution or resuspension with a carrier to form the injectable formulation for one or more of a subcutaneous, intramuscular, ocular, and intravenous injection.

15. The method of claim 14, wherein the carrier is non-aqueous and the injectable formulation is a suspension of the dried formulation and the injectable formulation comprises the dried formulation in a range of from 20% to 60% by weight.

16. The method of claim 14, further comprising storing the dried formulation for less than or equal to two years at a temperature in a range of 2° C. to 8° C.

17. The method of claim 14, wherein the carrier is aqueous and the injectable formulation is a solution of the dried formulation.

18. The method of claim 17, wherein the injectable formulation is for an intravenous injection and a concentration of bevacizumab in the injectable formulation is ≥0.1 mg/mL and ≤150 mg/mL.

19. The method of claim 17, wherein the injectable formulation is for one or more of an ocular injection, subcutaneous injection, and intramuscular injection, and a concentration of bevacizumab in the injectable formulation is in a range of from 100 mg/mL to 150 mg/mL.

20. The method of claim 14, wherein a tapped density of the dried formulation greater than or equal to 0.4 g/mL.
